(19) United States
(12) Patent Application Publication
(10) Pub. No.: US 2005/0107697 A1
(43) Pub. Date: May 19, 2005

(54) Contrast agent injection system

(75) Inventor: Ralph Berke, Zusmarshausen (DE)

Correspondence Address: SUGHRUE MION, PLLC, 2100 PENNSYLVANIA AVENUE, N.W., SUITE 800, WASHINGTON, DC, 20037, US

(73) Assignees: Ralph BERKE; Guido HILEKES

(21) Appl. No.: 10/989,287

(22) Filed: Nov. 17, 2004

(30) Foreign Application Priority Data
Dec. 23, 1998 (DE) 19859811.4

(51) Int. Cl. A61B 5/05

(52) U.S. Cl. 600/431

(57) ABSTRACT

Contrast agent injection system for injecting a contrast agent into a body, having at least a first injector 1 for delivering the contrast agent to a tube section 7 which is connected to the body, and at least a second injector 2 for delivering a rinsing fluid to the tube section 7, a control device 16 monitoring the delivery of the contrast agent through the tube section 7 and automatically controlling the delivery of the rinsing fluid by the second injector 2 after the delivery of the contrast agent has been terminated.

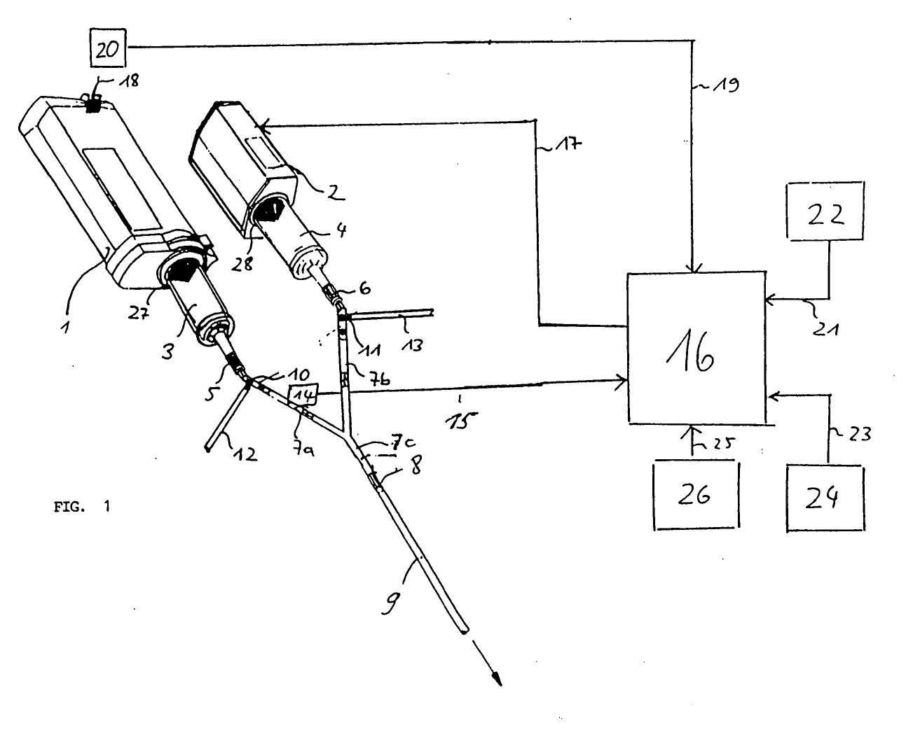

The invention relates to a contrast agent injection system for injecting a contrast agent into a body according to the preamble of patent claim 1.

DE 297 21 247 discloses an injection system for the combined bolus injection of contrast agent and sodium chloride solution. This injection system comprises two machine pressure syringes which are connected to the injection point on a patient's body in a parallel arrangement via a tube system. This permits automatic subsequent injection of a rinsing agent without having to change tube connections or open, close or switch over faucets in order to do so. In a first pressure syringe there is a contrast agent which is fed to the patient's body for diagnostic purposes, while a sodium chloride solution, for example, is provided as rinsing agent in a second pressure syringe. In particular in the field of computer tomography, the patient is injected with a contrast agent in order to show up vascularized tissue in X-ray investigations. So that the patient suffers as little stress as possible due to the quantity of contrast agent which is administered, and in order to reduce the risk of reactions by the body's defence system or complications, the volume of contrast agent which is to be injected is kept as small as possible. Furthermore, cost savings are made by minimizing the amount of relatively expensive contrast agent. However, on the other hand, the volume of contrast agent administered must be sufficiently large to avoid adversely affecting the quality of the diagnostics. The quantity of contrast agent administered must therefore be optimized as a critical factor when the injection is made. Subsequent injection with a rinsing agent, for example a sodium chloride solution, permits the remaining contrast agent located in the tube supplying the patient, and partially also the volume of contrast agent located in the patient's vein, to be utilized. The relatively cost-effective sodium chloride solution rinses out the injection path and thus significantly increases the yield of contrast agent.

The injection system described in DE 297 21 247 has the disadvantage that the automatic subsequent injection of the sodium chloride solution is initiated independently of the quantity of contrast agent actually flowing through the patient tube. There is thus no control over the quantity of contrast agent which actually gets into the patient's body. If only some of the contrast agent delivered by the contrast agent pressure syringe is supplied to the patient's body because of a faulty connection of the contrast agent injector to the tube, for example, a subsequent injection occurs even though sufficient contrast agent has not yet been fed into the patient's body. This excessively small quantity of contrast agent leads to a worsening of the quality during the X-ray diagnosis. Furthermore, the injection system described in DE 297 21 247 has a safety problem. If, for example, the engine controller of the machine contrast agent pressure syringe is imprecise or defective, it is possible, under certain circumstances, for an excessively large amount of contrast agent to be emitted into the patient tube without this being sensed and the inflow of contrast agent being promptly terminated. If excessively high quantities of contrast agent are supplied to the patient, this can result in damage to the patient's health.

The object of the present invention is therefore to provide a contrast agent injection system which is safe for the patient and which monitors the quantity of contrast agent fed to the patient's body. This object is achieved according to the invention by means of a contrast agent injection system having the features specified in patent claim 1.

Further advantageous refinements are specified in the subclaims.

The invention advantageously provides the possibility of subsequently retrofitting previously used single-piston injectors to form a dual-piston system. In this context it is advantageously possible to actuate the second or additional injector without direct electronic connection to the first injector, i.e. the second injector is activated by the control device without electronic connection to the first injector.

A preferred embodiment of the invention will be described below with reference to the appended FIGURE in order to explain the features which are essential to the invention.

The FIGURE shows a preferred embodiment of the contrast agent injection system according to the invention.

The contrast agent injection system has a first injector 1 for delivering a contrast agent and a second injector 2 for delivering a rinsing fluid. The injectors shown are single-piston injectors into which interchangeable pressure syringes 3, 4 are inserted. The contrast agent to be injected is located in the pressure syringe 3, and a rinsing fluid, for example a sodium chloride solution, is located in the pressure syringe 4. The two pressure syringes 3, 4 are each connected to an adapter 7 via connections 5, 6. The adapter 7 is connected via a connection 8 to a tube 9 and preferably has a Y-shaped structure. The tube 9 is connected to a blood vessel of the patient's body by means of an injection needle, for example. Two non-return valves 10, 11, preferably two double non-return valves 10, 11, are provided in the vicinity of the adapter 7 and/or at the output end of the respective filling unit, represented by the filling tubes 12, 13. The pressure syringe 3 can be filled with contrast agent via the filling tube 12 and the double non-return valve 10. The pressure syringe 4 can be filled with a rinsing agent via the filling tube 13 and the double non-return valve 11. According to the invention, a sensing device 14 is provided which senses the termination of the delivery of the contrast agent by the contrast agent injector 1. The sensing device 14 is preferably provided in the branch 7a of the adapter 7, as indicated in the FIGURE. When necessary, the sensing device 14 can also be provided at a different point.

In order to increase the monitoring reliability, a second sensing device (not shown) may additionally be provided, for example in the branch 7c of the Y-shaped adapter 7. The sensing device 14 is connected to a control device 16 via a line 15. The control device 16 controls, via a control line 17, the delivery of the rinsing fluid by the rinsing injector 2.

In a preferred embodiment, the contrast agent injector 1 has a status display 18, for example a status display lamp. This status display 18 indicates whether the contrast agent injector is ready to operate or whether the injection is terminated or interrupted. The control device 16 is connected via a line 19 to a status display detection device 20 which detects whether the status display lamp 18 is lit. The status display detection device 20 is, for example, an optocoupler or phototransistor which permits the status of the contrast agent injector 1 to be sensed in a galvanically isolated fashion.

In a preferred development, the control device 16 is connected via a line 21 to a contrast agent injection time calculating device 22 which contains a memory device for the contrast agent injection time. A contrast agent injection time can be stored in this memory device for the contrast agent inspection time. The contrast agent injection time calculating device 22 has a timer unit and outputs a signal to the control device 16 via the line 21 when the entire contrast agent injection time has expired.

In a further preferred development of the contrast agent injection system according to the invention, the control device 16 is connected via a line 23 to a contrast agent volume calculating device 24. The contrast agent volume calculating device 24 has a memory device for the contrast agent volume, in which the entire contrast agent volume which is to be delivered by the contrast agent injector 1 can be stored. The contrast agent volume calculating device 24 supplies a signal to the control device 16 via the line 23 when the total contrast agent volume is reached.

In a further preferred development of the contrast agent injection system according to the invention, the control device 16 is connected via a line 25 to an input device 26 by means of which, for example, the total contrast agent injection time or the total contrast agent volume can be input. The total contrast agent volume which is input is then stored in the memory device of the contrast agent volume calculating device 24. The total contrast agent injection time which is input is stored in the memory device of the contrast agent injection time calculating device 22.

The method of operation of the contrast agent injection system according to the invention will be described below. The sensing device 14 is preferably a fluid flow sensing device which senses the termination of the contrast agent flow in the section 7a. The sensing device 14 then outputs a signal to the control device 16 if no movement of fluid can be detected in the section 7a. A binary flag is then reset (FS=0), which indicates that there is no longer any flow in the section 7a in the control device 16.

The fluid flow sensing device 14 comprises, for example, a light-emitting unit and a light-receiving unit for sensing the light reflection caused by the fluid particles.

The sensing of the light reflection can be performed by an LED and phototransistor located in the same housing. The light emitted by the LED is reflected by the fluid particles in section 7a and received by the phototransistor. If no reflection occurs, it is detected that no fluid particles are present in the section 7a, and the flow is thus terminated.

Alternatively, a photoelectric barrier may also be provided, with the light signal emitted by a light-emitting unit being received more weakly at an associated light-receiving unit as a result of a fluid flowing in the tube, due to reflection from particles.

In a further embodiment, the fluid flow sensing device is composed of a sound-emitting unit and a sound-receiving unit for sensing the sound frequency shifts caused by the moving fluid particles.

In a preferred embodiment, the sensing device 14 is composed of an ultrasonic source which emits ultrasonic waves which are then reflected back by the fluid particles in the patient tube. The reflected ultrasonic waves are sensed by a sound-receiving unit, the received ultrasonic frequency shift being proportional to the velocity of flow in the section 7a.

In further embodiments, the sensing device 14 can, however, also utilize photoelectric, magnetic, inductive or mechanical effects in order to detect the flow of fluid in the section 7a.

As long as a flow of contrast agent is sensed in the section 7a, the sensing device 14 outputs a measurement signal, representing the flow, to the control device 16 via the line 15. From the known tube cross section of the section 7a and the sensed flow, the contrast agent volume calculating device 24 calculates the contrast agent volume which is being applied to the patient's body. If the applied contrast agent volume reaches the total contrast agent volume stored in the memory device of the contrast agent volume calculating device 24, the contrast agent volume calculating device 24 emits a signal which indicates that the total contrast agent volume has been reached. A binary volume flag VE in, for example, the control device 16 is reset (VE=0) by means of this signal.

The contrast agent injection time calculating device 22 supplies a time expiry signal to the control device 16 when the total contrast agent injection time is expired. A binary injection time flag ZE is then reset (ZE=0) in the control device 16.

The, if appropriate, additionally provided status display sensing device 20 supplies a sensing signal to the control device 16 via the line 19 if the injection by the contrast agent injector 1 is interrupted and the associated status lamp 18 is lit. A binary status display flag SS is then reset (SS=0) in the control device 16.

In one simple embodiment of the contrast agent injection system according to the invention, the control device 16 outputs an injection start signal to the rinsing agent injector 2 via the control line 17 when the flow flag FS is reset. The rinsing agent injector 2 then injects a rinsing agent, located in the pressure syringe 4, into the right-hand branch 7b of the adapter 7 via the connection 6, and presses the contrast agent still located in the patient tube 9 into the body.

In order to increase safety, in one preferred embodiment of the contrast agent injection system according to the invention the subsequent rinsing process is initiated only when all the binary monitoring flags FS, SS, VE, ZE have been reset. In this preferred embodiment, the control device 16 outputs an injection signal to the rinsing agent injector 2 only when the flow flag FS, if appropriate the status display flag SS, the contrast agent volume flag VE and the injection time flag ZE have been reset (FS=0, SS=0, VE=0, ZE=0). As a result, safety during the contrast agent injection is significantly increased because errors can also be detected.

The monitoring of a multiphase contrast agent injection in which various injection phases (injection steps), including programmed injection delays or pause times between the injection phases, are provided, can also be carried out by means of the control device 16.

After injection of the first injection phase, the status display flag SS and the flow flag FS are reset (FS=0; SS=0), but the total contrast agent volume flag VE and the injection time flag ZE are not reset (VE=1; ZE=1). These flags are also reset, and the subsequent injection of the rinsing agent by the rinsing agent injector 2 initiated, only when the entire contrast agent volume has been applied and the total contrast agent injection time has expired.

The error case, for example, of a bent patient tube 9 can also be detected by the control device 16 by means of the multiplicity of monitoring flags. If the patient tube 9 is unintentionally bent, the flow is interrupted and the flow flag FS is reset. The pressure in the tube system increases until a set pressure limit is reached, as a result of which the flow of contrast agent from the contrast agent injector 1 is automatically reduced or stopped. As a result, the contrast agent injection time is prolonged. After the total contrast agent injection time expires, the injection time flag ZE is reset, although the contrast agent injection has not yet been concluded. However, this does not also lead to an incorrect subsequent injection because in this case the total contrast agent volume has not yet been reached and the contrast agent volume flag VE has not yet been reset (VE=1). The contrast agent flag VE which has been set prevents the initiation of the subsequent injection.

In an incident or emergency in which the contrast agent injection is stopped by the contrast agent injector 1, the flow flag FS is reset but the other monitoring flags, i.e. the status display flag SS, the injection time flag ZE and the injection volume flag VE, which are all not yet reset, prevent the initiation of the subsequent injection. The control device 16 outputs, via the control line 17, a control signal for initiating the subsequent injection of the rinsing agent by the rinsing agent injector 2 only when all the monitoring flags FS, SS, ZE and VE have been reset.

Provision of various monitoring flags in the contrast agent injection system according to the invention considerably increases safety during the contrast agent injection. Furthermore, various error cases, such as bending of the patient tube or interruption of the contrast agent injection in an emergency can also be detected.

In a further embodiment (not shown), the rinsing agent subsequent injector is remote-controlled by the control device 16.

As is apparent from the FIGURE, there is no connection via an electrical connection between the contrast agent injector 1 and the subsequent injection controller 16, or the rinsing agent subsequent injector 2. The contrast agent injection system according to the invention is therefore extremely suitable for retrofitting in an already existing contrast agent injector 1. The contrast agent injector 1 simply has to be plugged onto the connection 5 of the Y-shaped adapter 7.

The refilling of the pressure syringes 3, 4 is preferably carried out as described below. The filling tubes 12, 13 are connected to a reservoir vessel. The pistons of the pressure syringes 3, 4 are then drawn back, as a result of which a partial vacuum arises in the pressure syringe chambers. The double non-return valves 10, 11 clear the path between the reservoir vessel and the pressure syringe, and the fluid is sucked into the pressure syringe from the reservoir vessel. At the same time, the double non-return valve 10, 11 ensures that the connection to the reservoir vessels remains closed during an injection process from the pressure syringes 3, 4 to the patient tube 9. After injection has taken place, the double non-return valve 10, 11 closes owing to the pressure equalization. Because the double non-return valve 10, 11 only permits a movement of injection fluid from the associated pressure syringe 3 or 4 to the patient's body, the body fluid originating from the patient's body is consequently also prevented from being sucked into the pressure syringe chamber. The same effect can be achieved by using a non-return valve in the patient tube 9.

The subsequent injection of the rinsing agent leads to a considerable saving in consumption of contrast agent. If the patient tube has, for example, a volume of approximately 2 ml and the relevant portion of the venous system has a volume of, for example, 10-15 ml, the contrast agent saving per patient is approximately 12 ml or 17 ml.

The contrast agent injection system according to the invention detects, by means of the flow sensing device near to the patient, whether too much contrast agent has passed into the patient's body. In addition, additional monitoring flags ensure that cases of errors during the injection of contrast agent are also detected.

In one preferred embodiment of the contrast agent injection system according to the invention, the control device 16 is connected via an interface which is already present on the supplementary injector 2, for example for manual triggering.

In a further preferred embodiment, the control device 16 has its own power supply which is independent of the power supply of the injectors 1, 2. This ensures additional safety during the delivery of the contrast agent.

In a further preferred embodiment of the contrast agent injection system according to the invention, the control device 16 controls a display device which indicates the status of the current injection. This display informs the viewer of, for example, how much contrast agent has already been applied or whether an error case has occurred.

In a further preferred embodiment of the invention, the pressure syringe mounting openings 27, 28 of the injectors 1, 2 have different cross sections, said cross sections respectively corresponding to a different mounting opening of the pressure syringes 3, 4. As a result, instances of confusion in which the pressure syringes are inserted into the incorrect injector are prevented. For example, the injector 1 has a round mounting opening for receiving the pressure syringe 3, while the supplementary injector 2 has a hexagonal opening for receiving the pressure syringe 4. The pressure syringe 3 is filled with contrast agent, while the pressure syringes 4 are filled with a rinsing agent. Erroneous confusion of the syringes is thus prevented.

1. Contrast agent injection system for injecting a contrast agent into a body, having at least a first injector for delivering the contrast agent to a tube connected to the body, and having at least a second injector for delivering a rinsing fluid to the tube, further comprising a control device being arranged independently form said injector, wherein said control device is connected with its output to said second injector for controlling said second injector, wherein the control device is connected with its input to a sensing device, said sensing or detecting device is sensing the fluid flow being produced by the said first injector within a subordinate tube section and producing a signal to said control device if no movement of fluid is detected, such that the control device activates the second injector, said tube section being connected to said first injector.

2. Contrast agent injection system for injecting a contrast agent into a body, having at least a first injector for delivering the contrast agent to a tube connected to the body, and having at least a second injector for delivering a rinsing fluid to the tube, further comprising a control device being arranged independently from said injector, wherein said control device is connected with its output to said second injector for controlling said second injector, wherein the control device is connected with its input to a detection device, which detects an optical signal from said first injector as a result of the interruption of the injection of said first injector and supplies a sensing signal to said control device, wherein the first injector comprises a device for producing a status display as an optical signal such that the control device activates the second injector when said detection device detects the interruption of the injection of the fluid flow in the tube section which is connected to said first injector.

3. Contrast agent injection system according to claim 1, wherein the fluid flow sensing device has a light-emitting unit and a light-receiving unit for sensing the light reflection caused by the fluid particles.

4. Contrast agent injection system according to claim 1, wherein the fluid flow sensing device has a sound-emitting unit and a sound-receiving unit for sensing the sound frequency shift caused by the moving fluid particles.

5. Contrast agent injection system according to claim 1, wherein the control device is connected to a contrast agent volume calculating device.

6. Contrast agent injection system according to claim 5, wherein the contrast agent volume calculating device comprises a contrast agent volume memory device in which the contrast agent volume located in the first injector is stored.

7. Contrast agent injection system for injecting a contrast agent into a body, having at least a first injector for delivering the contrast agent to a tube connected to the body, and having at least a second injector for delivering a rinsing fluid to the tube, further comprising a control device being arranged independently form said injector, wherein said control device is connected with its output to said second injector for controlling said second injector, wherein the control device is connected with its input to a sensing device, said sensing or detecting device is sensing the fluid flow being produced by the said first injector within a subordinate tube section and producing a signal to said control device if no movement of fluid is detected, such that the control device activates the second injector, said tube section being connected to said first injector, wherein the fluid flow sensing device has a light-emitting unit and a light-receiving unit for sensing the light reflection caused by the fluid particles within said tube section.

8. Contrast agent injection system for injecting a contrast agent into a body, having at least a first injector for delivering the contrast agent to a tube connected to the body, and having at least a second injector for delivering a rinsing fluid to the tube, further comprising a control device being arranged independently form said injector, wherein said control device is connected with its output to said second injector for controlling said second injector, wherein the control device is connected with its input to a sensing device, said sensing or detecting device is sensing the fluid flow being produced by the said first injector within a subordinate tube section and producing a signal to said control device if no movement of fluid is detected, such that the control device activates the second injector, said tube section being connected to said first injector, wherein the fluid flow sensing device has a sound-emitting unit and a sound-receiving unit for sensing the sound frequently shift caused by the moving fluid particles within said tube section.

9. Contrast agent injection system for injecting a contrast agent into a body, having at least a first injector for delivering the contrast agent to a tube connected to the body, and having at least a second injector for delivering a rinsing fluid to the tube, further comprising a control device being arranged independently form said injector, wherein said control device is connected with its output to said second injector for controlling said second injector, wherein the control device is connected with its input to a sensing device, said sensing or detecting device is sensing the fluid flow being produced by the said first injector within a subordinate tube section and producing a signal to said control device if no movement of fluid is detected, such that the control device activates the second injector, said tube section being connected to said first injector, wherein the fluid flow sensing device has a light-emitting unit and a light-receiving unit for sensing the light reflection caused by the fluid particles within said tube section and wherein the control device is connected to a contrast agent volume calculating device.

10. Contrast agent injection system for injecting a contrast agent into a body, having at least a first injector for delivering the contrast agent to a tube connected to the body, and having at least a second injector for delivering a rinsing fluid to the tube, further comprising a control device being arranged independently from said injector, wherein said control device is connected with its output to said second injector for controlling said second injector, wherein the control device is connected with its input to a detection device, which detects an optical signal from said first injector as a result of the interruption of the injection of said first injector and supplies a sensing signal to said control device, wherein the first injector comprises a device for producing a status display as an optical signal such that the control device activates the second injector when said detection device detects the interruption of the injection of the fluid flow in the tube section which is connected to said first injector, wherein the control device is connected to a contrast agent injection time calculating device.

11. Contrast agent injection system for injecting a contrast agent into a body, having at least a first injector for delivering the contrast agent to a tube connected to the body, and having at least a second injector for delivering a rinsing fluid to the tube, further comprising a control device being arranged independently from said injector, wherein said control device is connected with its output to said second injector for controlling said second injector, wherein the control device is connected with its input to a detection device, which detects an optical signal from said first injector as a result of the interruption of the injection of said first injector and supplies a sensing signal to said control device, wherein the first injector comprises a device for producing a status display as an optical signal such that the control device activates the second injector when said detection device detects the interruption of the injection of the fluid flow in the tube section which is connected to said first injector, wherein the control device is connected to a contrast agent injection time calculating device, wherein the contrast agent injection time is stored and the contrast agent injection time calculating device has a contrast agent injection time memory device in which the contrast agent injection time is stored.

12. Contrast agent injection system for injecting a contrast agent into a body, having at least a first injector for delivering the contrast agent to a tube connected to the body, and having at least a second injector for delivering a rinsing fluid to the tube, further comprising a control device being arranged independently from said injector, wherein said control device is connected with its output to said second injector for controlling said second injector, wherein the control device is connected with its input to a sensing device, said sensing or detecting device is sensing the fluid flow being produced by the said first injector within a subordinate tube section and producing a signal to said control device if no movement of fluid is detected, such that the control device activates the second injector, said tube section being connected to said first injector, said control device being connected to an input device for inputting the contrast agent volume and the contrast agent injection time.

13. Contrast agent injection system for injecting a contrast agent into a body, having at least a first injector for delivering the contrast agent to a tube connected to the body, and having at least a second injector for delivering a rinsing fluid to the tube, further comprising a control device being arranged independently from said injector, wherein said control device is connected with its output to said second injector for controlling said second injector, wherein the control device is connected with its input to a sensing device, said sensing or detecting device is sensing the fluid flow being produced by the said first injector within a subordinate tube section and producing a signal to said control device if no movement of fluid is detected, such that the control device activates the second injector, said tube section being connected to said first injector, wherein the first injector and the second injector are connected via an adapter to said tube, further comprising valves for filling the injectors said valves being controlled via the control device.

14. Contrast agent injection system for injecting a contrast agent into a body, having at least a first injector for delivering the contrast agent to a tube connected to the body, and having at least a second injector for delivering a rinsing fluid to the tube, further comprising a control device being arranged independently from said injector, wherein said control device is connected with its output to said second injector for controlling said second injector, wherein the control device is connected with its input to a sensing device, said sensing or detecting device is sensing the fluid flow being produced by the said first injector within a subordinate tube section and producing a signal to said control device if no movement of fluid is detected, such that the control device activates the second injector, said tube section being connected to said first injector, wherein the fluid flow sensing device has a light-emitting unit and a light-receiving unit for sensing the light reflection caused by the fluid particles and wherein the injectors are single-piston or mulitpiston injectors comprising interchangeable pressure syringes which are arranged in mounting openings of said injectors, said mounting openings having different mounting openings corresponding to the associated pressure syringes.

15. Contrast agent injection system for injecting a contrast agent into a body, having at least a first injector for delivering the contrast agent to a tube connected to the body, and having at least a second injector for delivering a rinsing fluid to the tube, comprising a control device being arranged independently from said injector, wherein said control device is connected with its output to said second injector for controlling said second injector, the control device being connected with its input to a sensing device, said sensing device being provided for sensing the fluid flow within a subordinate tube section, said fluid flow being produced by said first injector, such that the control device activates the second injector, if the sensing device detects an interruption of the fluid flow within the tube section, which is connected to said first injector, and outputs a signal to the control device, wherein the control device is connected to a contrast agent volume calculating device, and the contrast agent volume calculating device has a contrast agent volume memory device in which the contrast agent volume located in the first injector is stored.

16. Contrast agent injection system for injecting a contrast agent into a body, having at least a first injector for delivering the contrast agent to a tube connected to the body, and having at least a second injector for delivering a rinsing fluid to the tube, comprising a control device being arranged independently form said injector, wherein said control device is connected with its output to said second injector for controlling said second injector, wherein the control device is connected with its output said second injector for controlling said second injector and with its input to a detection device which detects an optical signal from said first injector as a result of the interruption of the injection of said first injector, wherein the first injector comprises a device for producing a status display as an optical signal such that the control device activates the second injector when said detection device detects the interruption of the injection of the fluid flow in the tube section which is connected to said first injector, wherein the control device is connected to a contrast agent volume calculating device, and the contrast agent volume calculating device has a contrast agent volume memory device in which the contrast agent volume located in the first injector is stored.

17. Contrast agent injection system for injecting a contrast agent into a body, having at least a first injector for delivering the contrast agent to a tube connected to the body, and having at least a second injector for delivering a rinsing fluid to the tube, comprising a control device being arranged independently form said injector, wherein said control device is connected with its output to said second injector for controlling said second injector, wherein the control device is connected with its output said second injector for controlling said second injector, that the control device is connected with its input to a detection device which detects an optical signal from said first injector as a result of the interruption of the injection of said first injector, wherein the first injector comprises a device for producing a status display as an optical signal such that the control device activates the second injector when said detection device detects the interruption of the injection of the fluid flow in the tube section which is connected to said first injector, wherein the control device is connected to a contrast agent volume calculating device, the control device is connected to a contrast agent injection time calculating device, and the contrast agent injection time calculating device has a contrast agent injection memory device in which the contrast agent injection time is stored.
